(19) United States
(12) Patent Application Publication
(10) Pub. No.: US 2022/0019395 A1
(43) Pub. Date: Jan. 20, 2022

(54) INFORMATION PROCESSING APPARATUS

(71) Applicant: Wacom Co., Ltd., Saitama (JP)

(72) Inventors: Kaelin CHANG, Portland, OR (US); Jun KADOWAKI, Saitama (JP); Isao MARUOKA, Saitama (JP); Markus WEBER, Duesseldorf (DE); Philipp Daniel SCHONGEN, Duesseldorf (DE); Toru EGUCHI, Saitama (JP)

(21) Appl. No.: 17/449,787

(22) Filed: Oct. 1, 2021

Related U.S. Application Data
(63) Continuation of application No. PCT/JP2019/044088, filed on Nov. 11, 2019.

(30) Foreign Application Priority Data
Apr. 5, 2019 (JP) 2019-072566

(51) Int. Cl. G06F 3/14; G06F 3/044; G06F 3/046; A61B 5/00; B43K 29/08

(52) U.S. Cl. CPC G06F 3/14; G06F 3/044; B43L 19/0043; A61B 5/7425; B43K 29/08; G06F 3/046

(57) ABSTRACT

Provided is an information processing apparatus including a display device including a display screen, a communication circuit which, in operation, exchanges information with plural portable terminals, and a control circuit connected to the display device and the communication circuit, in which the display screen includes a display area having a plurality of partial display areas in a plurality of separate regions of the display screen, each of the partial display areas being associated with a different one of the portable terminals, and the control circuit, in operation, displays, in each partial display area of the partial display areas, information communicated via the communication circuit with the one of the portable terminals associated with the partial display area.

### BACKGROUND

### Technical Field

This disclosure relates to an information processing apparatus that includes a display device and a communication circuit and that is capable of communicating with a plurality of portable terminals.

### Background Art

Information, e.g., electronic mails, is often transmitted or received via a communication circuit. When electronic mails are transmitted, for example, contents (i.e., an outgoing message) of each electronic mail to be transmitted are typically displayed on a display screen together with information of a destination, a subject, etc., and are transmitted after being checked. When electronic mails are received, for example, contents (i.e., an incoming message) of each electronic mail to be received are typically displayed on the display screen together with information of a source, a subject, etc., and are checked.

However, displaying items of information to be transmitted or received, one after another, as described above may involve inconvenience in a case where, for example, three or more people exchange electronic mails with respect to the same topic, for example.

To mitigate such inconvenience, Japanese Patent Laid-Open No. 2002-351789 has proposed a system that provides display areas for a plurality of electronic mails on a display screen and enables a user to perform a transmission/reception process and, at the same time, check messages, destinations, senders, subjects, etc., of a plurality of electronic mails.

However, the system of Japanese Patent Laid-Open No. 2002-351789 is configured such that items of information of a plurality of electronic mails having any destinations or sources that are selected by the user when necessary can be displayed on the display screen at the same time.

Accordingly, even in a case where electronic mails are exchanged among a limited number of people, such as members of a family or members of a group in a company, selection needs to be made as necessary when an electronic mail is to be transmitted or when reception information is to be checked, which involves inconvenience.

### BRIEF SUMMARY

This disclosure has been conceived of to provide an information processing apparatus that is able to solve the above problem.

In order to solve the abovementioned problem, provided is an information processing apparatus that includes a display device including a display screen, a communication circuit which, in operation, exchanges information with a plurality of portable terminals, and a control circuit connected to the display device and the communication circuit. The display screen includes a display area having a plurality of partial display areas in a plurality of separate regions of the display screen, each of the partial display areas being associated with a different one of the portable terminals. The control circuit, in operation, displays, in each partial display area of the partial display areas, information communicated via the communication circuit with the one of the portable terminals associated with the partial display area.

In the information processing apparatus having the above configuration, the plurality of partial display areas formed in separate regions of the display area of the display screen of the display device, and each of the partial display areas has a different portable terminal registered so as to be associated therewith. That is, each partial display area is associated with a particular portable terminal set in advance.

In addition, the control circuit performs control such that, in each of the partial display areas, information (transmission information and/or reception information) communicated with the portable terminal associated with that partial display area is displayed.

### BRIEF DESCRIPTION OF THE DRAWINGS

FIG. 1 is a diagram for explaining outlines of an example of a configuration of an information processing apparatus according to an embodiment of this disclosure and a mode of use thereof;

FIG. 2 is an exploded perspective view illustrating an example of a hardware configuration of the information processing apparatus according to the embodiment;

FIG. 3 is a diagram for explaining an example of a position indicator used in conjunction with the information processing apparatus according to the embodiment;

FIG. 4 is a diagram illustrating an example of a configuration of an electronic circuit of the information processing apparatus according to the embodiment;

FIG. 5 illustrates an example of table information provided in the information processing apparatus according to the embodiment;

FIG. 6 is a block diagram illustrating an example of a configuration of a portable terminal that performs communication with the information processing apparatus according to the embodiment;

FIG. 7 is a block diagram illustrating an example of a configuration of a wearable terminal that communicates with the portable terminal that performs communication with the information processing apparatus according to the embodiment;

FIG. 8 is a diagram illustrating a portion of a flowchart for explaining an example of a flow of processing of the information processing apparatus according to the embodiment;

FIG. 9 is a diagram illustrating a portion of the flowchart for explaining the example of the flow of the processing of the information processing apparatus according to the embodiment;

FIG. 10 is a diagram illustrating a portion of the flowchart for explaining the example of the flow of the processing of the information processing apparatus according to the embodiment;

FIG. 11 is a diagram illustrating a portion of the flowchart for explaining the example of the flow of the processing of the information processing apparatus according to the embodiment;

FIG. 12 is a diagram illustrating a portion of a flowchart for explaining an example of a flow of processing of a portable terminal that performs communication with the information processing apparatus according to the embodiment;

FIG. 13 is a diagram illustrating a portion of the flowchart for explaining the example of the flow of the processing of the portable terminal that performs communication with the information processing apparatus according to the embodiment;

FIG. 14 is a diagram illustrating a portion of the flowchart for explaining the example of the flow of the processing of the portable terminal that performs communication with the information processing apparatus according to the embodiment; and

FIG. 15 is a diagram illustrating a flowchart for explaining an example of a flow of processing of a portable terminal that performs communication with the information processing apparatus according to the embodiment.

### DETAILED DESCRIPTION

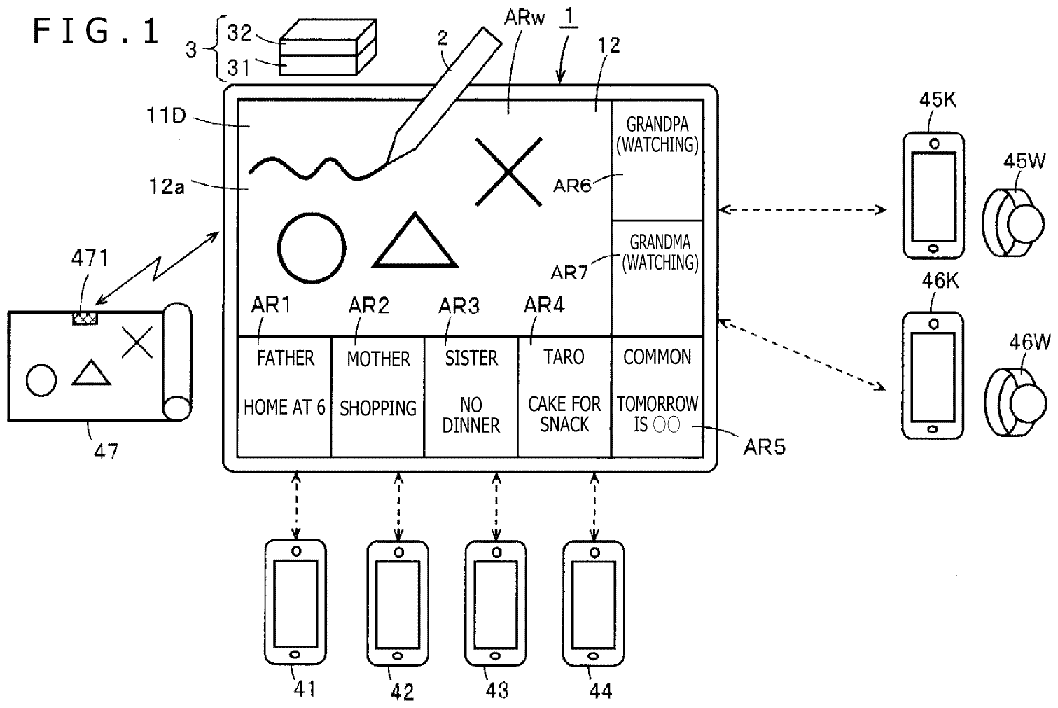

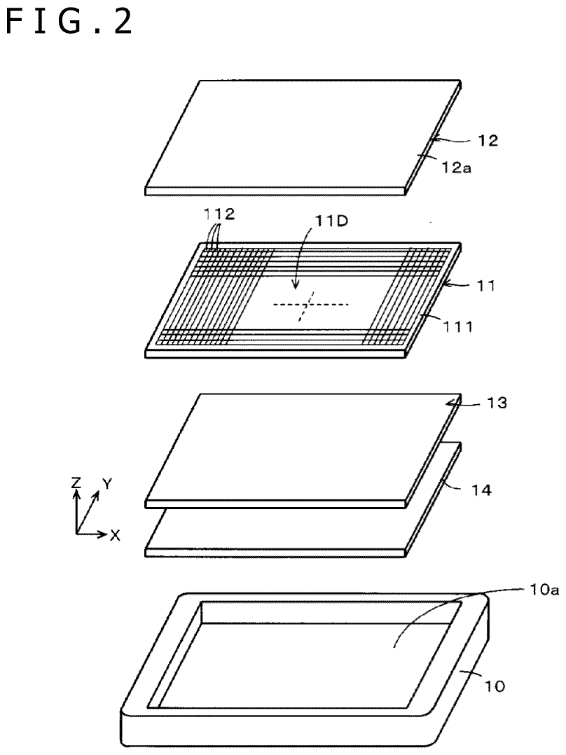

Hereinafter, an information processing apparatus according to an embodiment of this disclosure will be described with reference to the accompanying drawings. FIG. 1 is a diagram for explaining outlines of an example of a configuration of an information processing apparatus 1 according to an embodiment of this disclosure and an example of a mode of use of the information processing apparatus 1. FIG. 2 is an exploded perspective view illustrating the example of the configuration of the information processing apparatus 1.

As illustrated in FIG. 2, the information processing apparatus 1 in this example includes a case 10, and further includes a display device 11, a transparent panel 12 that forms a cover member disposed thereon so as to cover an upper portion (i.e., a side on which a display screen lies) of a display screen of the display device 11, a position detection sensor 13 disposed below (i.e., on a rear side of) the display device 11, and a control circuit board 14 that forms an electronic circuit assembly of the information processing apparatus 1, which are stacked one upon another and are housed in the case 10.

The case 10 is made of, for example, synthetic resin, and has a recessed portion 10a to house the display device 11, the transparent panel 12, the position detection sensor 13, and the control circuit board 14.

The display device 11 is formed by a large-sized flat display, measuring, for example, 40 or more inches diagonally, and formed by a liquid crystal display, an organic electroluminescence (EL) display, or the like. The display device 11 includes a display board 111 and a display screen 11D that is disposed thereon and that has a large number of display pixels 112 arranged in an X-axis direction (i.e., a horizontal direction) and in a Y-axis direction (i.e., a vertical direction) perpendicular to the X-axis direction.

In this example, the transparent panel 12 is formed by a transparent material, e.g., a transparent acrylic panel, which allows writing and drawing using ink to be made thereon through a marker pen portion of a position indicator 2 and which allows such writing and drawing to be erased through an eraser function member 3. In this example, the transparent panel 12 has the same size as that of the display screen 11D of the display device 11, and a front surface 12a of the transparent panel 12 is used as an operation surface for position indication by the position indicator 2.

In this example, the position detection sensor 13, which is of an electromagnetic induction type, is disposed below (i.e., on the rear side of) the display device 11 such that a position detection area of the position detection sensor 13 precisely coincides with the display screen 11D. In this example, the detection area of the position detection sensor 13 where the position indicated by the position indicator 2, which will be described below, is detected has the same size as that of a display area of the display screen 11D of the display device 11, and the display device 11 and the position detection sensor 13 are disposed such that the two areas precisely coincide with each other.

In addition, the control circuit board 14 is disposed on the rear side of the position detection sensor 13. The control circuit board 14 is electrically connected to each of the display device 11 and the position detection sensor 13.

Further, the information processing apparatus 1 according to this embodiment is accompanied by the position indicator 2 with a marker, which is capable of indicating a position through electromagnetic inductive coupling with the position detection sensor 13 and is also capable of making ink writing on the front surface 12a of the transparent panel 12; and the eraser function member 3, which is capable of erasing a writing mark of the ink writing made with the marker-attached position indicator 2 from the front surface 12a of the transparent panel 12 and is also capable of sending an instruction to delete corresponding writing data to the position detection sensor 13.

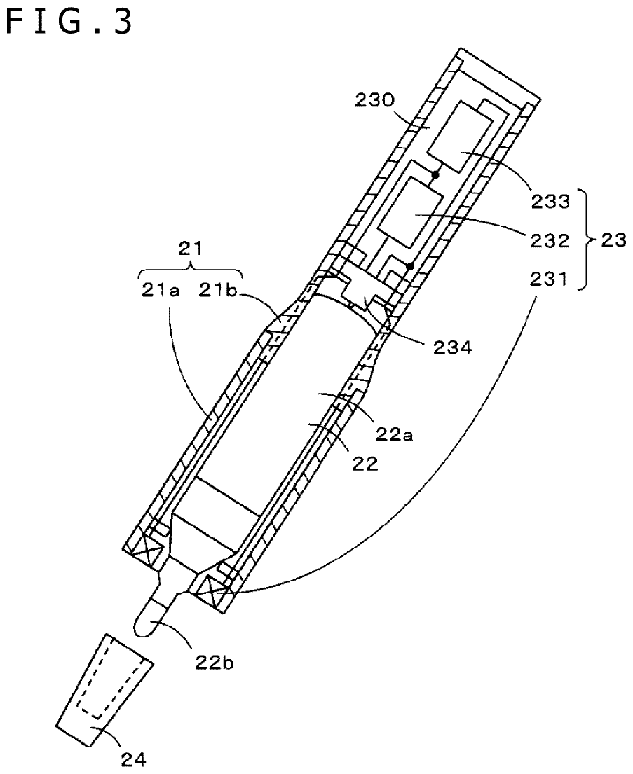

FIG. 3 illustrates an example of a configuration of the marker-attached position indicator 2. In this example, the marker-attached position indicator 2 includes a case 21 including a tip-side case 21a and a rear-side case 21b coupled to each other through screwing, and further includes a marker pen body 22 and an interaction portion 23 that interacts with, i.e., exchanges a signal with, the position detection sensor 13, which are disposed in the case 21.

The marker pen body 22 is configured to allow ink stored in an ink reservoir portion 22a to be fed to a pen point member 22b made of, for example, felt, and is similar to a felt pen on the market. A cap 24 is attached to the pen point member 22b.

The interaction portion 23 includes a resonant circuit including a coil 231 and capacitors 232 and 233. As illustrated in FIG. 3, the coil 231 is disposed in the vicinity of the pen point member 22b of the marker pen body 22 in the tip-side case 21a. In addition, the capacitors 232 and 233 are disposed on a printed circuit board 230 provided on a rear side of the ink reservoir portion 22a of the marker pen body 22 in the rear-side case 21b.

In addition, a switch 234 is disposed on the printed circuit board 230 so as to be opposite to a tail portion of the ink reservoir portion 22a of the marker pen body 22. The switch 234 is turned on and off through axial displacement of the marker pen body 22 to control whether or not the capacitor 233 is to be connected with the resonant circuit. When ink writing is made with the pen point member 22b of the marker pen body 22, if a pressure applied to the pen point member 22b of the marker pen body 22 is equal to or smaller than a predetermined value, the switch 234 is off, so that the capacitor 233 is not connected with the resonant circuit, whereas if a pressure greater than the predetermined value is applied to the pen point member 22b, the switch 234 is on, so that the capacitor 233 is connected with the resonant circuit, resulting in a change in resonance frequency thereof.

The change in the resonance frequency due to the turning on and off of the switch 234 can be used, for example, to give an instruction as to whether or not, when ink writing formed on the front surface 12a of the transparent panel 12 through the marker pen body 22 has been erased with the eraser function member 3, writing data corresponding to the ink writing stored is to be deleted together. For example, instructions may be given to cause writing data corresponding to ink writing formed through the marker pen body 22 when the switch 234 is in an off state to be deleted together, and to cause writing data corresponding to ink writing formed through the marker pen body 22 when the switch 234 is in an on state with a pressure being applied to the pen point member 22b of the marker-attached position indicator 2 to remain as stored data without being deleted.

The eraser function member 3 includes an eraser portion 31 including a member to erase, through friction, a writing mark of ink writing formed on the front surface 12a of the transparent panel 12 through the marker-attached position indicator 2; and an interaction portion 32 for interacting with the position detection sensor 13 to delete corresponding writing data. The eraser portion 31 is made of a well-known fiber material or the like. The interaction portion 32 includes a resonant circuit including a coil and a capacitor and is provided in a holder that holds the eraser portion 31.

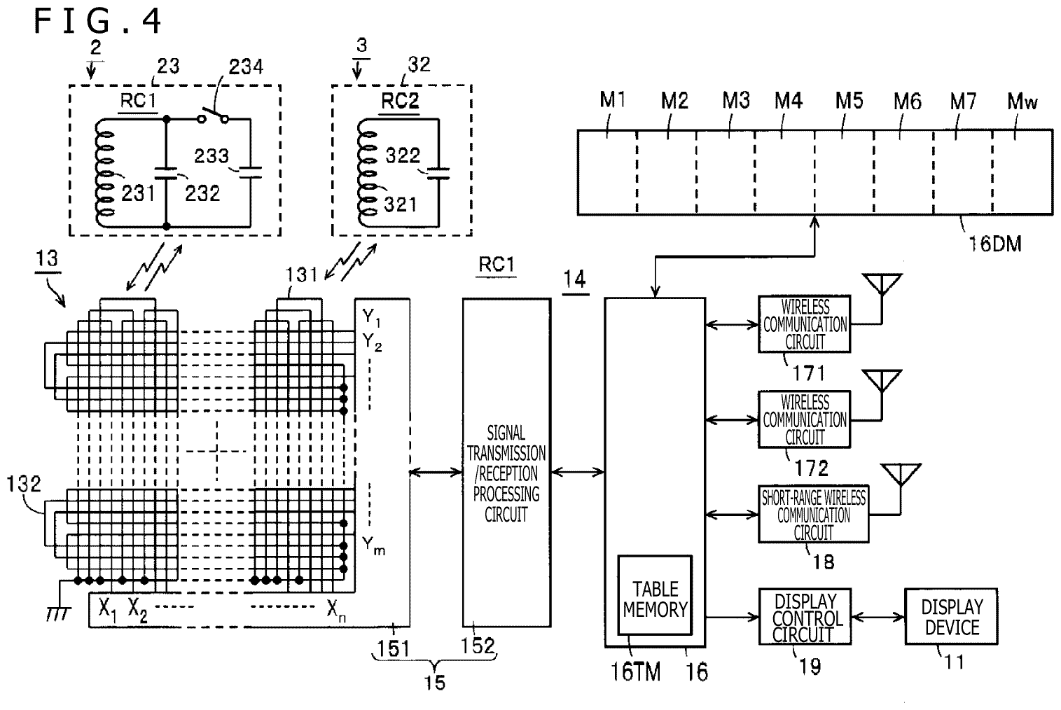

FIG. 4 is a diagram illustrating an example of a configuration of an electronic circuit formed on the abovementioned control circuit board 14, including how the electronic circuit is connected to the position detection sensor 13 and the display device 11. In addition, FIG. 4 also illustrates how the position detection sensor 13 interacts with each of the marker-attached position indicator 2 and the eraser function member 3.

In the upper left of FIG. 4, an example of a circuit configuration of the interaction portion 23 of the marker-attached position indicator 2 and an example of a circuit configuration of the interaction portion 32 of the eraser function member 3 are illustrated. Specifically, the interaction portion 23 of the marker-attached position indicator 2 has a resonant circuit RC1 in which the coil 231 and the capacitor 232 are connected in parallel and the capacitor 233 is additionally connected in parallel therewith through the switch 234. The resonance frequency of the resonant circuit RC1 varies depending on whether the switch 234 is off or on.

The interaction portion 32 of the eraser function member 3 has a resonant circuit RC2 in which a coil 321 and a capacitor 322 are connected in parallel. The resonance frequency of the parallel resonant circuit RC2 is configured to be different from the resonance frequency of the resonant circuit RC1.

Each of the resonant circuit RC1 of the marker-attached position indicator 2 and the resonant circuit RC2 of the eraser function member 3 exchanges signals (i.e., interacts) with the position detection sensor 13 through electromagnetic inductive coupling therewith.

Meanwhile, as illustrated in FIG. 4, the control circuit board 14 is provided with a position detection circuit 15, a process control circuit 16, two wireless communication circuits 171 and 172 in this example, a short-range wireless communication circuit 18, and a display control circuit 19. In this example, the short-range wireless communication circuit 18 performs wireless communication complying with the Bluetooth (registered trademark) standard.

In this example, the position detection circuit 15 includes selection circuitry 151 connected to the position detection sensor 13 and a signal transmission/reception processing circuit 152 connected to each of the selection circuitry 151 and the process control circuit 16.

In this example, the position detection sensor 13 includes position detection coils including an X-axis direction loop coil group 131 and a Y-axis direction loop coil group 132 in a stack. In addition, each of loop coils of the X-axis direction loop coil group 131 and the Y-axis direction loop coil group 132 is connected to the selection circuitry 151. The selection circuitry 151 performs a selection process based on control signals from a control circuit included in the signal transmission/reception processing circuit 152, thereby selecting the loop coils in the two loop coil groups 131 and 132 one after another, causing a signal from the signal transmission/reception processing circuit 152 to be transmitted to the resonant circuit of the eraser function member 3 or the position indicator 2, which will be described later, and receiving a signal returned from the resonant circuit to cause the signal to be supplied to the signal transmission/reception processing circuit 152.

Further, in the signal transmission/reception processing circuit 152 of the position detection circuit 15, positions on the position detection sensor 13 which are simultaneously indicated when ink writing is made with the marker-attached position indicator 2 are detected from among positions on the position detection sensor 13 at which return signals received from the resonant circuit RC2 of the interaction portion 23 of the marker-attached position indicator 2 through electromagnetic coupling are detected, and a result of this detection is supplied to the process control circuit 16 as information (hereinafter referred to as writing data) of the writing input made with the marker-attached position indicator 2.

In addition, in the signal transmission/reception processing circuit 152 of the position detection circuit 15, positions on the position detection sensor 13 for which an erasure instruction is given through the eraser function member 3 are detected from among positions on the position detection sensor 13 at which return signals received from the resonant circuit RC2 of the interaction portion 32 of the eraser function member 3 through electromagnetic coupling are detected, and a result of this detection is supplied to the process control circuit 16 as information of erasure positions inputted with the eraser function member 3.

The process control circuit 16 stores the writing data acquired from the signal transmission/reception processing circuit 152 in a memory, and performs a process of displaying the acquired writing data on the display screen 11D of the display device 11. In addition, when the information of the inputted erasure positions has been acquired from the signal transmission/reception processing circuit 152, the process control circuit 16 performs a process of deleting corresponding writing data from the memory.

In addition, in this embodiment, the process control circuit 16 has a function of communicating with an external portable terminal on the basis of information from the signal transmission/reception processing circuit 152 of the position detection circuit 15. Further, in this embodiment, the process control circuit 16 is configured to be also capable of communicating with an external portable terminal regardless of information from the signal transmission/reception processing circuit 152 of the position detection circuit 15. Furthermore, the process control circuit 16 is also configured to control display on the display screen 11D during this communication.

Entities with which the information processing apparatus 1 according to this embodiment communicates are limited to entities set in advance. In addition, in the information processing apparatus 1 according to this embodiment, partial display areas are set in the display screen 11D with each partial display area assigned to a separate one of the previously set entities to communicate with, and the information processing apparatus 1 is configured to manage communication associated with each of the partial display areas.

The information processing apparatus 1 according to this embodiment is installed in, for example, a living room of a house in which a family of a plurality of members, such as a father, a mother, a sister, and Taro, lives together, for example, and can be used by any of the members who is in the house and any of the members who is out of the house. The information processing apparatus 1 according to this embodiment is capable of communicating with portable terminals that are carried by the respective members when the members go out. In this example, a high-performance mobile phone terminal called a smart phone, for example, is used as such a portable terminal. Any of the members who is in the house is able to use the information processing apparatus 1 according to this embodiment as a communication terminal. In the description of this example, electronic mail communication is used as an example of communication.

As illustrated in FIG. 1, in the information processing apparatus 1 according to this embodiment, seven partial display areas AR1, AR2, AR3, AR4, AR5, AR6, and AR7 are set in the display area of the display screen 11D of the display device 11. The partial display areas AR1 to AR7 may be set in advance in the information processing apparatus 1, or may optionally be set by a user. Each of the partial display areas AR1 to AR7, which is formed by a rectangular region, can be set by specifying positions of, for example, a top left corner and a bottom right corner of the partial display area.

In this embodiment, the display area of the display screen 11D and the position detection area of the position detection sensor 13 lie one on top of the other, and display positions in the display area and positions in the position detection area are associated with each other. Thus, if the position of an area in one of the display area and the position detection area is identified, the position of a corresponding area in the other one of the display area and the position detection area can be obtained. Accordingly, once the partial display areas are set, partial input areas corresponding to the respective partial display areas are automatically computed and set in the position detection area of the position detection sensor 13.

Note that, in a case where any partial display area is set, a setting of a partial input area in the detection area of the position detection sensor 13 may be accepted to set a partial display area corresponding to that partial input area. Note that the number of partial display areas may be one, is not limited to the number of the example illustrated in the figure, and may be any value. Also note that the size of each partial display area may be set optionally, and that, when a plurality of partial display areas are set, the partial display areas may vary in size. In addition, also note that the shape of an area set as a partial display area is not limited to a rectangular shape as in the example illustrated in the figure and may be circular or a shape of any other polygon.

Once the setting of the partial display areas is completed, items of communication address information of portable terminals 41 to 44, 45K, and 46K, i.e., items of information of electronic mail addresses in this example, are set so as to be associated with the respective partial display areas. Various methods are applicable as a method for setting the information of the electronic mail addresses. Examples of such methods include writing input using the marker-attached position indicator 2, for example, and a method of causing an electronic mail to be transmitted from each of the portable terminals 41 to 44, 45K, and 46K to the information processing apparatus 1 with the corresponding partial display area being selected, for example, and having the electronic mail of the transmitter set so as to be associated with the corresponding partial display area.

Next, a name of each of the partial display areas AR1 to AR7 is set through writing input using the marker-attached position indicator 2, for example. Further, in this embodiment, a special use may be set for the partial display areas AR1 to AR7 in a case where any of the partial display areas AR1 to AR7 is used not simply for transmission and reception of electronic mails by a particular person (i.e., for an individual use) but for another special purpose. Such a special use may be selected from a list of uses applicable to the information processing apparatus 1, the list being displayed in advance. In this example, a common use is set as the use of the partial display area AR5, and a watching use, which is helpful in monitoring the state of health of a grandfather or grandmother living at another place, is set for each of the partial display areas AR6 and AR7.

Here, an area for which the common use is set is used as a common area that is used in common by users of all the portable terminals registered and set for the individual use, and as described later, when writing has been made on the common area with use of the marker-attached position indicator 2, information of the writing is transmitted to all the portable terminals registered and set for the individual use. Meanwhile, an example of the watching use is as follows. A grandfather and a grandmother to be watched over wear a wearable terminal, such as a smart watch, and biological information regarding the person to be watched over acquired in the wearable terminals is regularly transmitted via electronic mails through the portable terminals, and the received biological information is displayed on the corresponding partial display areas AR6 and AR7.

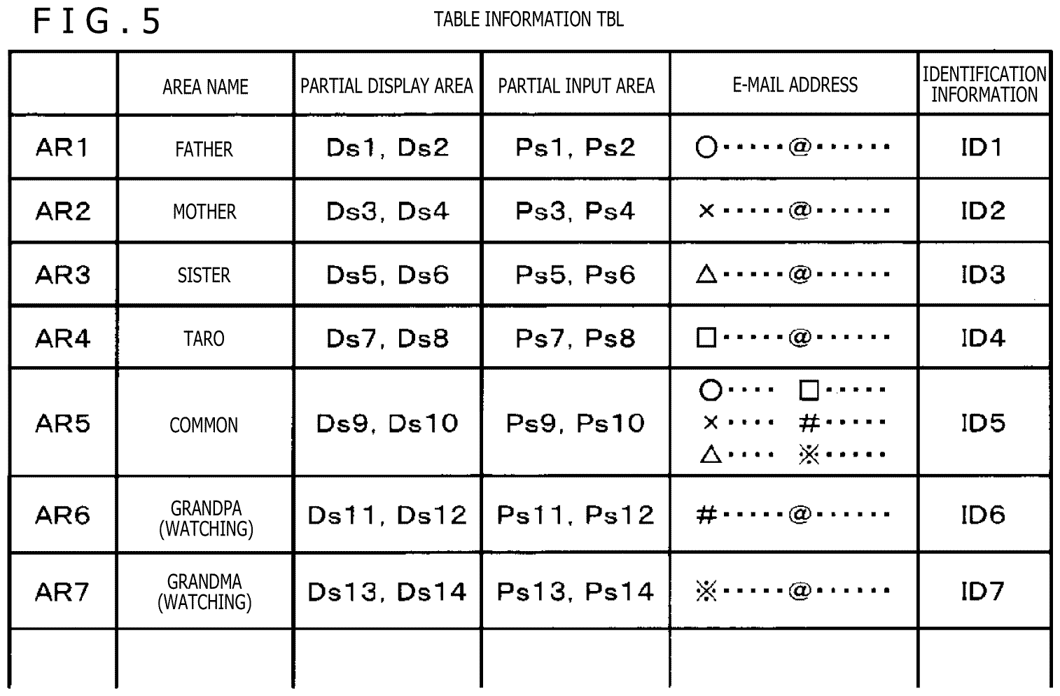

Once the partial display areas, the partial input areas, and the corresponding electronic mail addresses are set as described above, the process control circuit 16 stores the associated items of information in a table memory 16TM contained therein in this example. FIG. 5 illustrates an example of table information TBL, which is contents stored in the table memory 16TM.

Area names in the table information TBL in the table memory 16TM are indicated for the respective partial display areas AR1 to AR7 as illustrated in FIG. 1. Note that the name “Common,” which specifies a special use, is assigned as the area name of the partial display area AR5. Meanwhile, “(Watching),” which is indicated with enclosing brackets in the area names of the partial display areas AR6 and AR7, indicates that the watching use is set.

Two values (e.g., Ds1 and Ds2) of a partial display area represent, respectively, the position of the display pixel at the left corner of the partial display area in the display area and the position of the display pixel at the right corner of the partial display area. Meanwhile, two values (e.g., Ps1 and Ps2) of a partial input area represent, respectively, the position of a left corner of the partial input area in the detection area of the position detection sensor 13 and the position of a right corner of the partial input area.

The electronic mail addresses are, as described above, electronic mail addresses of the portable terminals associated with the respective partial display areas AR1 to AR7. Specifically, as illustrated in FIG. 1, an electronic mail address of the portable terminal 41 carried by the father is set, registered, and stored for the partial display area AR1; an electronic mail address of the portable terminal 42 carried by the mother is set, registered, and stored for the partial display area AR2; an electronic mail address of the portable terminal 43 carried by the sister is set, registered, and stored for the partial display area AR3; and an electronic mail address of the portable terminal 44 carried by Taro is set, registered, and stored for the partial display area AR4. In addition, the electronic mail addresses of the portable terminals 41 to 44 carried respectively by the father, the mother, the sister, and Taro are set, registered, and stored for the partial display area AR5 for the common use. Further, an electronic mail address of the portable terminal 45K, which communicates with a wearable terminal 45W worn by the grandfather and collects and transmits biological information regarding the grandfather, is set, registered, and stored for the partial display area AR6. Further, an electronic mail address of the portable terminal 46K, which communicates with a wearable terminal 46W worn by the grandmother and collects and transmits biological information regarding the grandmother, is set, registered, and stored for the partial display area AR7.

Further, in this example, identification information (identification (ID)) is assigned to each of the partial display areas AR1 to AR7.

In this embodiment, an application program for performing electronic mail communication with the information processing apparatus 1 according to this embodiment is installed in advance on each of the portable terminals 41 to 44 for the individual use. Meanwhile, an application program for performing communication for the watching use in conjunction with the information processing apparatus 1 is installed in advance on each of the portable terminals 45K and 46K for the watching use.

As illustrated in FIG. 1, a remaining display area ARw, which is not associated with any portable terminal, is present in the display area of the display screen 11D of the display device 11 of the information processing apparatus 1 in this example. In this example, the remaining display area ARw is used as an area for any writing input. Further, the information processing apparatus 1 has, in this example, a function of performing short-range wireless communication with a position detection sensor-attached drawing paper apparatus 47, and performs control such that drawing data (painting data) sent from the position detection sensor-attached drawing paper apparatus 47 is displayed on the remaining display area ARw.

The position detection sensor-attached drawing paper apparatus 47 includes a position detection sensor (omitted from illustration) disposed on a rear side of a sheet of paper for painting and a short-range wireless communication circuit 471, and has a function of transmitting painting data detected by the position detection sensor to the information processing apparatus 1 through the short-range wireless communication circuit 471. The short-range wireless communication circuit 471 performs wireless communication complying with the Bluetooth (registered trademark) standard, for example.

Note that apparatuses which perform short-range wireless communication with the information processing apparatus 1 are not limited to the position detection sensor-attached drawing paper apparatus 47, and may include any other apparatus having a function of transmitting painting data to the information processing apparatus 1, such as a drawing tablet apparatus.

In this embodiment, the wireless communication circuit 171 and the wireless communication circuit 172 are provided for the process control circuit 16 to enable communication with the portable terminals 41 to 44 and the portable terminals 45K and 46K described above. The plurality of wireless communication circuits 171 and 172 are provided to make it possible to respond to communication requests from the portable terminals 45K and 46K for the watching use as quickly and securely as possible, since regular communication is required for the watching use, and there may sometimes be urgency with respect to information for the watching use.

In addition, the short-range wireless communication circuit 18 for communicating with the short-range wireless communication circuit 471 of the above-described position detection sensor-attached drawing paper apparatus 47 is connected to the process control circuit 16.

Further, as illustrated in FIG. 4, an information storage memory 16DM is connected to the process control circuit 16. In this example, a storage area of the information storage memory 16DM is divided into a plurality of storage areas M1 to M7 and Mw associated with the partial display areas AR1 to AR7 and the remaining display area ARw, respectively.

In addition, in each of the storage areas M1 to M7 and Mw, information displayed on a corresponding one of the partial display areas AR1 to AR7 and the remaining display area ARw and writing data of position indication input made with the marker-attached position indicator 2 onto a corresponding one of the partial input areas and the remaining display area ARw are stored, and the stored items of information are managed separately in the respective storage areas. In this example, in the storage areas M1 to M7 and Mw, the display information of the partial display areas AR1 to AR7 and the writing data of the corresponding partial input areas are stored so as to be managed separately, and the display information of the remaining display area ARw and the corresponding writing data are stored so as to be managed separately.

As illustrated in FIG. 4, the process control circuit 16 is connected to the display device 11 via the display control circuit 19, and controls the display on each of the partial display areas AR1 to AR7 and the remaining display area ARw of the display screen 11D of the display device 11, according to the information stored in the corresponding one of the storage areas M1 to M7 and Mw of the information storage memory 16DM.

Boldface characters in the partial display areas AR4 and AR5 and figures drawn in a bold line in the remaining display area ARw on the display screen 11D in FIG. 1 represent ink writing made with the marker-attached position indicator 2, and writing data thereof is stored in the information storage memory 16DM. However, at the time of input of the writing, the ink writing is simply made on the front surface 12a of the transparent panel 12 and is not displayed as display information on the display screen of the display device 11.

### <Outline of Processing Operation of Process Control Circuit 16>

The process control circuit 16 stores information received from the portable terminals 41 to 44 and the portable terminals 45K and 46K, through the wireless communication circuit 171 or 172, respectively in the storage areas M1 to M4, M6, and M7, which correspond to the partial display areas AR1 to AR4, AR6, and AR7 with which the respective portable terminals are associated. Further, the process control circuit 16 generates display information corresponding to the received information, and causes the generated information to be displayed on the partial display areas associated with the respective portable terminals. Note that, in the case where ink writing on the front surface 12a of the transparent panel 12 made with the marker-attached position indicator 2 is present in an overlapping manner on any of the partial display areas AR1 to AR4, making the display information thereon hard to see, the display information can be made easier to see by erasing the ink writing with the eraser function member 3.

In this case, when the process control circuit 16 has received information from the portable terminals 45K and 46K associated with the partial display areas AR6 and AR7 set to be used for the “watching use,” the process control circuit 16 also performs a process of causing the received information to be displayed on the partial display areas AR6 and AR7 in such a manner as to facilitate recognition of the state of health of the people to be watched over, for example, by making the change over time of the biological information regarding the person to be watched over easier to see.

In addition, in this embodiment, the process control circuit 16 has a function of accepting information of a display information acquisition request from the portable terminals 41 to 44 and returning display information displayed on the display screen 11D thereto. In this case, in this embodiment, the display information acquisition request from the portable terminals 41 to 44 may include information (hereinafter referred to as a specification-attached request) specifying a partial display area other than the corresponding partial display area, information (a common request) specifying the common area, or information (a whole area request) specifying a request for acquisition of the display information of the whole display area of the display screen 11D.

Note that, in the information processing apparatus 1 according to this embodiment, the display information of each area may include the ink writing made on the front surface 12a of the transparent panel 12 with the pen point member 22b of the marker pen body 22 of the marker-attached position indicator 2. This is because, when ink writing has been made on the front surface 12a of the transparent panel 12 with the pen point member 22b of the marker pen body 22 of the marker-attached position indicator 2, writing data corresponding to the ink writing is detected by the position detection circuit 15 through the position detection sensor 13 and is stored, and the writing data can thus be used as display information corresponding to the ink writing in the information processing apparatus 1.

When the process control circuit 16 has received information of a display information acquisition request without such specification information attached thereto, the process control circuit 16 returns, to the requesting portable terminal, display information displayed on the partial display area associated with the requesting portable terminal. In addition, when the process control circuit 16 has received a specification-attached request, the process control circuit 16 returns, to the requesting portable terminal, display information displayed on the specified partial display area, in addition to the display information displayed on the partial display area with which the requesting portable terminal is associated, or in place of the display information displayed on the partial display area with which the requesting portable terminal is associated.

In addition, when the process control circuit 16 has received a whole area request, the process control circuit 16 returns, to the requesting portable terminal, the display information of the whole display area of the display screen 11D.

In addition, when the process control circuit 16 has received, from the signal transmission/reception processing circuit 152, writing data based on input of positions indicated by the marker-attached position indicator 2 and detected by the position detection circuit 15, the process control circuit 16 identifies an area, in the detection area of the position detection sensor 13, at which this writing data is inputted. Then, the process control circuit 16 stores the received writing data in the storage area in the information storage memory 16DM which corresponds to the identified area, and, when it has been determined that the identified area corresponds to any of the partial display areas AR1 to AR4 and the partial display areas AR6 and AR7, transmits the received writing data to the portable terminal set to be associated with the identified partial display area.

When the identified partial display area is the partial display area AR5 for which the “common use” is set, the process control circuit 16 transmits the received writing data to all the portable terminals 41 to 44 set to be associated with the partial display areas AR1 to AR4. In the case of this “common use,” the received writing data may be transmitted to the portable terminals 45K and 46K as well. Note that, in this embodiment, writing data of writing input onto the remaining display area ARw is not transmitted to the portable terminals 41 to 44 at the time of writing.

In addition, the information processing apparatus 1 according to this embodiment is configured such that, when writing input has been made at any of the partial display areas AR1 to AR4 for the individual use through position indication input using the marker-attached position indicator 2, it is possible to make a request (forwarding request) to transmit (forward) the corresponding writing data not only to the portable terminal set to be associated with the partial display area at which the writing input has been made, but also to the portable terminal set to be associated with any other partial display area.

The process control circuit 16 also has a function of, when issuance of an instruction for a forwarding request has been detected on the basis of information from the signal transmission/reception processing circuit 152, performing a process of forwarding the received writing data to the portable terminal specified by the forwarding request.

### <Example of Hardware Configuration of Portable Terminals 41 to 44, 45K, and 46K>

In this example, each of the portable terminals 41 to 44, 45K, and 46K is a common high-performance mobile phone terminal called a smart phone. However, as mentioned above, an application program for performing communication as described above with the information processing apparatus 1 is installed in advance on each of the portable terminals 41 to 44, 45K, and 46K.

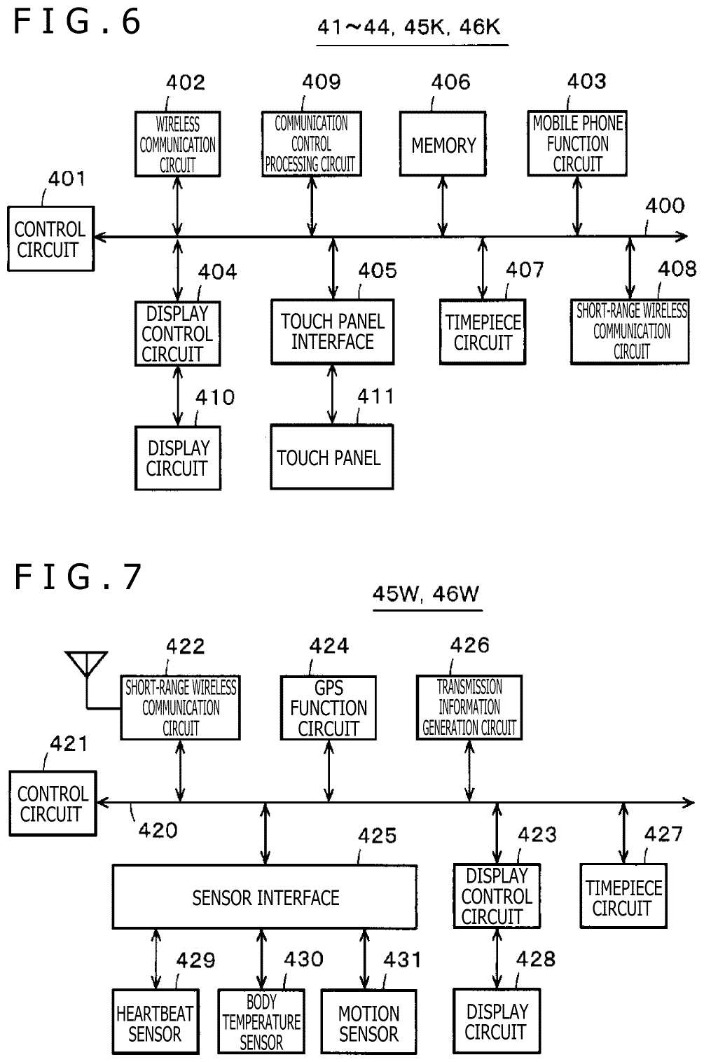

Each of the portable terminals 41 to 44, 45K, and 46K has substantially the same hardware configuration as illustrated in FIG. 6. In the following description, for the sake of convenience in description, the hardware configuration will be described with reference to the example of a configuration of the portable terminal 41.

As illustrated in FIG. 6, the portable terminal 41 is configured such that a wireless communication circuit 402, a mobile phone function circuit 403, a display control circuit 404, a touch panel interface 405, a memory 406, a timepiece circuit 407, a short-range wireless communication circuit 408, and a communication control processing circuit 409 are connected to a control circuit 401 including a computer, via a system bus 400. A display device 410 including, for example, a liquid crystal display (LCD) is connected to the display control circuit 404. A touch panel 411 is connected to the touch panel interface 405. The touch panel 411 is disposed on a rear side of a display screen of the display device 410 so as to overlap with the display screen.

The wireless communication circuit 402 is used to perform wireless communication with another communication terminal, such as the information processing apparatus 1, and is capable of performing telephone communication as implemented by a common mobile phone, through the mobile phone function circuit 403. Information received and information inputted through the touch panel 411 or the like are stored in the memory 406. The timepiece circuit 407 provides information as to a date and time. In this example, the short-range wireless communication circuit 408 performs wireless communication complying with the Bluetooth (registered trademark) standard, and is used to communicate with, for example, the wearable terminal 45W through wireless connection therewith.

In this example, the communication control processing circuit 409 is a functional block representing a circuit implemented by the application program for performing wireless communication as described above with the information processing apparatus 1. An electronic mail address of the information processing apparatus 1 is stored, as a destination address of an electronic mail, in the communication control processing circuit 409, and the communication control processing circuit 409 performs processing control for the communication with the information processing apparatus 1 described above.

In the case of each of the portable terminals 41 to 44, the communication control processing circuit 409 performs a process for transmission by the user of an electronic mail to the information processing apparatus 1, and also performs a process of requesting the information processing apparatus 1 to transmit the display information displayed on the display screen 11D thereof and a process of receiving information transmitted from the information processing apparatus 1.

Meanwhile, in the case of each of the portable terminals 45K and 46K, the communication control processing circuit 409 performs a process of transmitting the biological information regarding the person to be watched over to the information processing apparatus 1 at predetermined intervals, in addition to or in place of the above-described processes that are performed in the case of each of the portable terminals 41 to 44.

Accordingly, in the case of each of the portable terminals 45K and 46K, the communication control processing circuit 409 performs monitoring for a timing at which the biological information regarding the person to be watched over is to be transmitted to the information processing apparatus 1, and, when it is determined that such a timing has come, sends a request for acquisition of the biological information to the wearable terminal 45W or 46W, and transmits, to the information processing apparatus 1, the biological information sent in response to the request.

### <Example of Hardware Configuration of Wearable Terminals 45W and 46W>

In this example, each of the wearable terminals 45W and 46W is a type of terminal (i.e., a smart watch) which is worn by a person as a wrist watch. The wearable terminals 45W and 46W have configurations similar to each other, and have a configuration as illustrated in FIG. 7. In the following description, for the sake of convenience in description, the configuration will be described as the configuration of the wearable terminal 45W.

As illustrated in FIG. 7, the wearable terminal 45W is configured such that a short-range wireless communication circuit 422, a display control circuit 423, a global positioning system (GPS) function circuit 424, a sensor interface 425, a transmission information generation circuit 426, and a timepiece circuit 427 are connected to a control circuit 421 including a computer, via a system bus 420. A display device 428 including, for example, an LCD is connected to the display control circuit 423.

The short-range wireless communication circuit 422 is used to perform wireless communication with the short-range wireless communication circuit 408 of the portable terminal 45K. The GPS function circuit 424 is used to detect a current position of the target person wearing the wearable terminal 45W.

In this example, a heartbeat sensor 429, a body temperature sensor 430, and a motion sensor 431 are connected to the sensor interface 425. The heartbeat sensor 429, the body temperature sensor 430, and the motion sensor 431 respectively detect a heartbeat, a body temperature, and a body motion of the target person wearing the wearable terminal 45W. Note that, needless to say, a sensor for acquiring another type of biological information may be connected to the sensor interface 425 as necessary.

The transmission information generation circuit 426 generates transmission information regarding the target person wearing the wearable terminal 45W from biological information from the heartbeat sensor 429, the body temperature sensor 430, and the motion sensor 431 via the sensor interface 425, position information from the GPS function circuit 424, and information as to a date and time at which the biological information has been acquired from the timepiece circuit 427.

The control circuit 421 transmits the transmission information generated by the transmission information generation circuit 426 to the portable terminal 45K in response to a request for acquisition of the biological information from the portable terminal 45K.

### <Description of Example of Flow of Processing of Information Processing Apparatus 1>

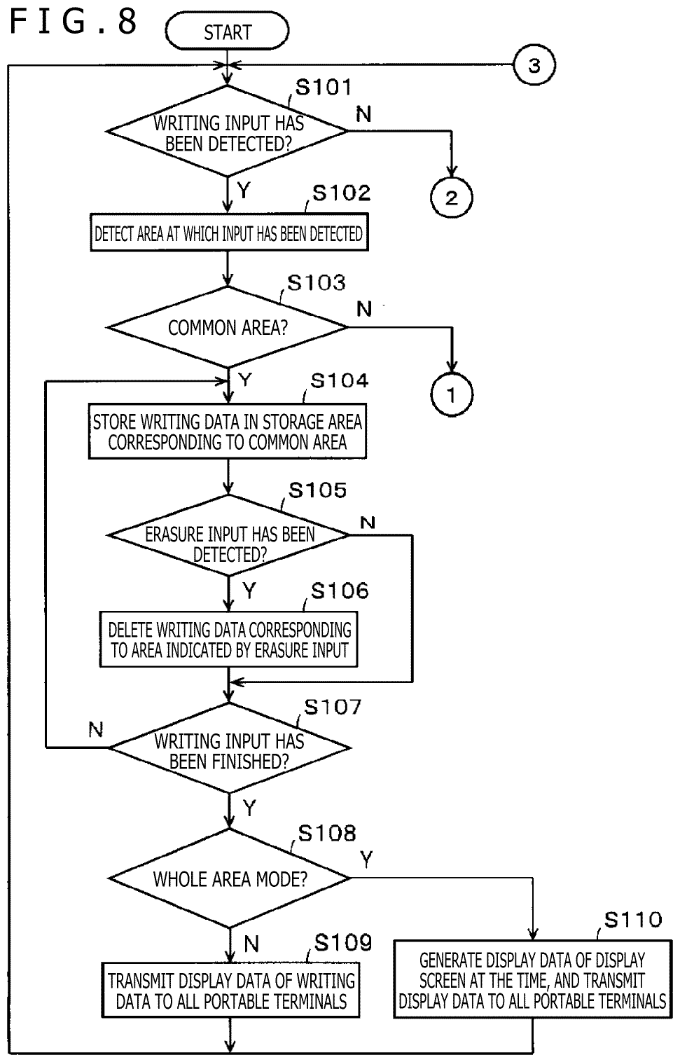

FIGS. 8 to 11 are diagrams for explaining an example of a flow of a processing operation performed by the process control circuit 16 in the information processing apparatus 1 according to this embodiment.

The process control circuit 16 detects whether or not writing input by position indication input onto the position detection sensor 13 with use of the marker-attached position indicator 2 has been detected (S101). If it is determined at S101 that writing input has been detected, the process control circuit 16 detects the area at which this writing input has been detected in the detection area of the position detection sensor 13 (S102).

Then, the process control circuit 16 refers to the table information TBL illustrated in FIG. 5, and determines whether or not the detected area is the common area for which the “common use” is set, i.e., the partial display area AR5 in this example (S103), and, if it is determined that the detected area is the partial display area AR5 set as the common area, the process control circuit 16 stores writing data based on the detected writing input in the storage area M5, corresponding to the partial display area AR5, in the information storage memory 16DM, together with information as to whether or not the writing data can be deleted, the information varying depending on the resonance frequency of the detected writing input (S104).

Next, the process control circuit 16 determines whether or not erasure input by the eraser function member 3 has been detected (S105), and, if it is determined that erasure input has been detected, the process control circuit 16 deletes writing data corresponding to an area indicated by the erasure input (S106). Here, because pen writing input has not been finished yet, the writing data corresponding to the area indicated by the erasure input is deleted regardless of the information as to whether or not the writing data can be deleted. At a time after the writing input is finished, it is determined whether or not the writing data is to be deleted together, on the basis of the information as to whether or not the writing data can be deleted, when an operation of erasing ink writing has been performed using the eraser function member 3.

If it is determined at S105 that erasure input has not been detected, or after the writing data corresponding to the area indicated by the erasure input is deleted at S106, the process control circuit 16 determines whether or not the writing input using the marker-attached position indicator 2 has been finished (S107), and, if it is determined that the writing input has not been finished yet, the process control circuit 16 returns control to S104. Here, this determination as to whether or not the writing input has been finished at S107 may be made on the basis of clear instruction input via writing input by a user of the marker-attached position indicator 2, or it may be determined that the writing input has been finished when writing input has not been detected at the area detected at S102 for a predetermined period of time or longer.

If it is determined at S107 that the writing input using the marker-attached position indicator 2 has been finished, the process control circuit 16 determines whether or not a “whole area mode” is on (S108). If it is determined at S108 that the “whole area mode” is not on, the process control circuit 16 transmits display data of the writing data for the partial input area corresponding to the partial display area AR5 for the common use at which the writing input has been detected to all the portable terminals set to be associated with the partial display area AR5, e.g., the portable terminals 41 to 44 (S109). Then, the process control circuit 16 returns control to S101.

Meanwhile, if it is determined at S108 that the “whole area mode” is on, the process control circuit 16 transmits, not only the display data of the writing data for the partial display area AR5, which is the common area, at which the writing input has been detected, but also display data displayed on the display area of the display screen 11D to all the portable terminals set to be associated with the partial display area AR5, which is the common area, e.g., the portable terminals 41 to 44 (S110). Then, the process control circuit 16 returns control to S101.

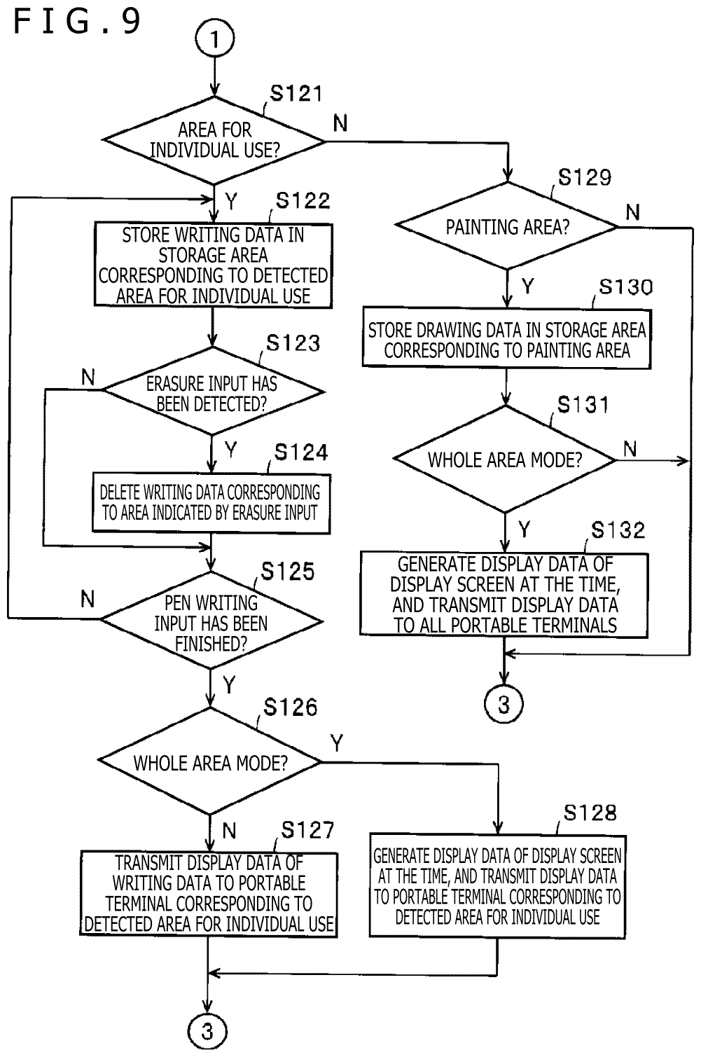

Meanwhile, if it is determined at S103 that the area at which the writing input has been detected is not the common area, i.e., the partial display area AR5 in this example, the process control circuit 16 refers to the table information TBL illustrated in FIG. 5, and determines whether or not the area at which the writing input has been detected is a partial input area corresponding to any of the partial display areas AR1 to AR4 for the individual use (S121 in FIG. 9). Note that, at S121, the partial display areas AR6 and AR7 for the watching use may be added to the partial display areas for the individual use.

If it is determined at S121 that the area at which the writing input has been detected is a partial input area corresponding to any of the partial display areas AR1 to AR4 for the individual use, the process control circuit 16 stores the detected writing data in the storage area corresponding to the detected partial input area in the information storage memory 16DM, together with the information as to whether or not the writing data can be deleted, the information varying depending on the resonance frequency of the detected writing input (S122). Then, the process control circuit 16 determines whether or not erasure input by the eraser function member 3 has been detected (S123), and, if it is determined that an erasure input has been detected, the process control circuit 16 deletes writing data corresponding to an area indicated by the erasure input (S124). Here, because pen writing input has not been finished yet, the writing data corresponding to the area indicated by the erasure input is deleted regardless of the information as to whether or not the writing data can be deleted.

If it is determined at S123 that erasure input has not been detected, or after the writing data corresponding to the area indicated by the erasure input is deleted at S124, the process control circuit 16 determines whether or not the writing input using the marker-attached position indicator 2 has been finished (S125), and, if it is determined that the writing input has not been finished yet, the process control circuit 16 returns control to S122.

If it is determined at S125 that the writing input using the marker-attached position indicator 2 has been finished, the process control circuit 16 determines whether or not the “whole area mode” is on (S126). If it is determined at S126 that the “whole area mode” is not on, the process control circuit 16 transmits display data of the writing data for the partial input area corresponding to the partial display area for the individual use at which the writing input has been detected to the portable terminal set to be associated with this partial display area for the individual use (S127). Then, the process control circuit 16 returns control to S101 in FIG. 8.

Meanwhile, if it is determined at S126 that the “whole area mode” is on, the process control circuit 16 transmits, not only the display data of the writing data for the partial input area corresponding to the partial display area for the individual use at which the writing input has been detected, but also the display data displayed on the display area of the display screen 11D to the portable terminal set to be associated with this partial display area for the individual use (S128). Then, the process control circuit 16 returns control to S101 in FIG. 8.

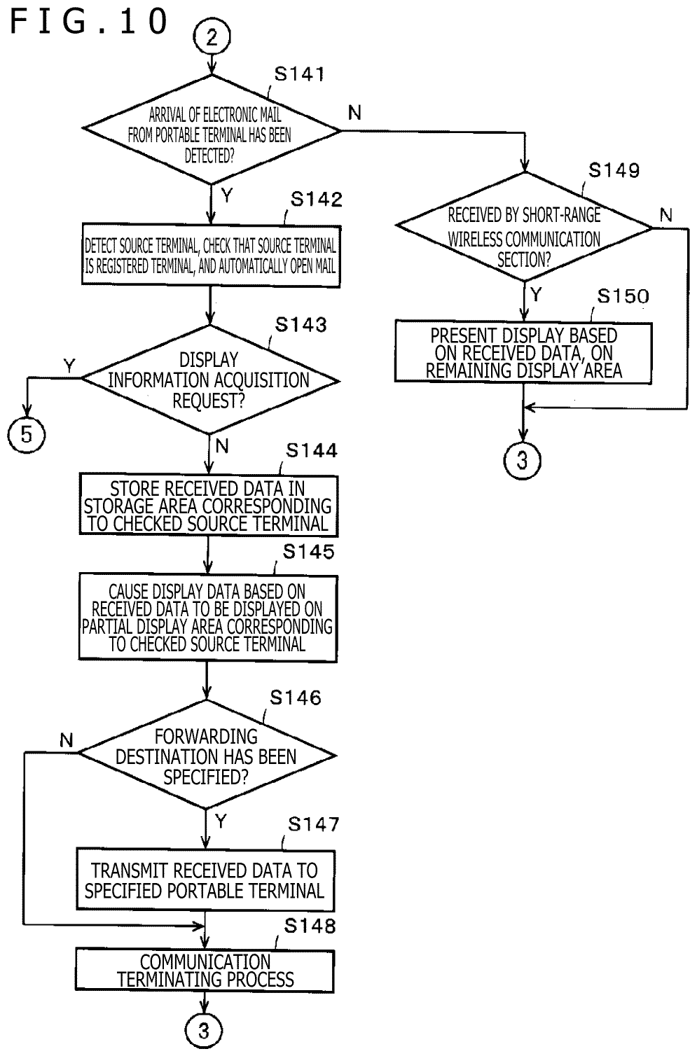

Next, if it is determined at S101 in FIG. 8 that writing input has not been detected, the process control circuit 16 determines whether or not arrival of an electronic mail has been detected (S141 in FIG. 10). If it is determined at S141 that arrival of an electronic mail has been detected, the process control circuit 16 refers to the table information TBL illustrated in FIG. 5 with use of information of a source electronic mail address to check whether the source is any of the registered portable terminals 41 to 44, 45K, and 46K, and automatically opens the electronic mail if it is found that the source is any of the registered portable terminals (S142).

Then, it is determined whether or not the received electronic mail includes a display information acquisition request (S143). If it is determined at S143 that no display information acquisition request is included therein, the process control circuit 16 stores received data in the storage area corresponding to the partial display area corresponding to the source portable terminal found at S142 in the information storage memory 16DM (S144).

Next, the process control circuit 16 causes display data based on the received data to be displayed on the partial display area corresponding to the source portable terminal found at S142 (S145).

Next, the process control circuit 16 determines whether or not the received electronic mail includes a specification of a forwarding destination (S146). If it is determined that no specification of a forwarding destination is included therein, the process control circuit 16 performs a communication terminating process (S148), and then returns control to S101 in FIG. 8. Meanwhile, if it is determined that a specification of a forwarding destination is included in the received electronic mail, the received data is transmitted to the specified portable terminal among the portable terminals set to be associated with the partial display areas AR1 to AR4 (S147). Here, if the specification of a forwarding destination indicates “common,” it means that the plurality of portable terminals associated with the partial display area AR5, which is the common area, have been specified as forwarding destinations, and the received data is transmitted to the other portable terminals except the portable terminal that has transmitted the received data. Then, the process control circuit 16 performs the communication terminating process (S148), and thereafter returns control to S101 in FIG. 8.

Meanwhile, if it is determined at S141 that arrival of an electronic mail from a portable terminal has not been detected, the process control circuit 16 determines whether or not drawing data (painting data) sent from the position detection sensor-attached drawing paper apparatus 47 has been received via the short-range wireless communication circuit 18 (S149), and, if it is determined that such drawing data has been received, the process control circuit 16 displays the received drawing data (painting data) on the remaining display area ARw of the display screen 11D (S150). Then, the process control circuit 16 returns control to S101 in FIG. 8. Meanwhile, if it is determined at S149 that such drawing data (painting data) has not been received, the process control circuit 16 returns control to S101 in FIG. 8, skipping S150.

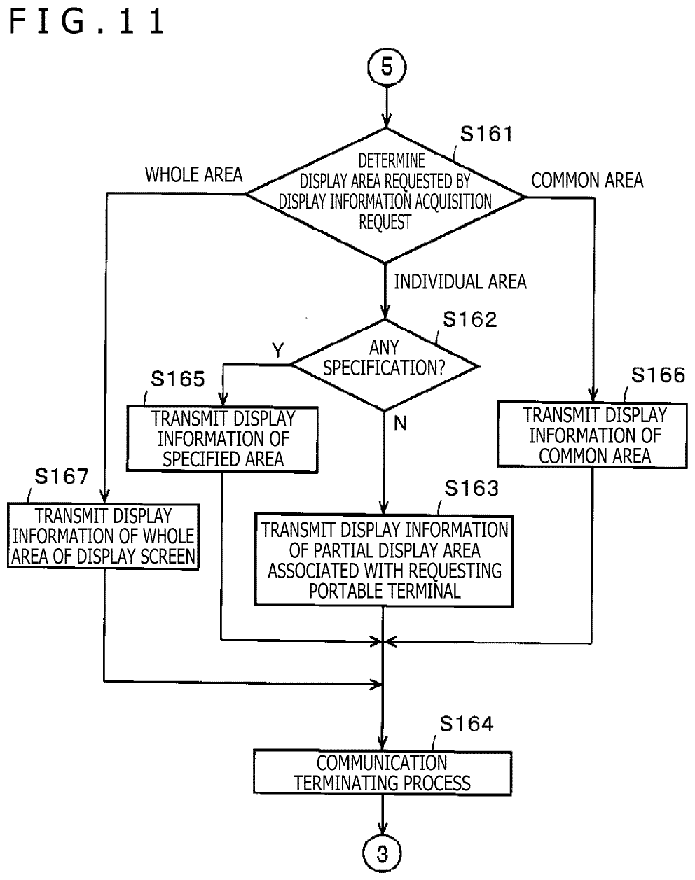

Meanwhile, if it is determined at S143 that the received electronic mail includes a display information acquisition request, the process control circuit 16 identifies an area in the display area of the display screen 11D to which the display information requested by the electronic mail including the display information acquisition request belongs (S161 in FIG. 11).

If it is determined at S161 that the display information of any of the partial display areas AR1 to AR4 for the individual use is requested, the process control circuit 16 determines whether or not any specification about the partial display areas AR1 to AR4 is made (S162). This specification may be a specification of an area name, such as “father,” “mother,” “sister,” or “Taro,” or may be a specification of identification information specifying any of the partial display areas. On the mobile phone, this specification can be performed by selecting an item from a displayed list of the partial display areas for the individual use. The number of forwarding destinations specified may be one or more than one.

If it is determined at S162 that no such specification is made, the process control circuit 16 transmits the display information of the partial display area associated with the requesting portable terminal to the requesting portable terminal (S163). Next, the process control circuit 16 performs the communication terminating process (S164), and then returns control to S101 in FIG. 8.

Meanwhile, if it is determined at S162 that such a specification is made, the process control circuit 16 transmits the display information of the specified partial display area to the requesting portable terminal (S165). Next, the process control circuit 16 performs the communication terminating process (S164), and then returns control to S101 in FIG. 8.

Meanwhile, if it is determined at S161 that the display information of the partial display area AR5, which is the common area, is requested, the process control circuit 16 transmits the display information of the partial display area AR5 to the requesting portable terminal (S166). Next, the process control circuit 16 performs the communication terminating process (S164), and then returns control to S101 in FIG. 8.

Meanwhile, if it is determined at S161 that the display information of the whole display area of the display screen 11D is requested, the process control circuit 16 transmits the display information displayed on the display screen 11D to the requesting portable terminal (S167). Next, the process control circuit 16 performs the communication terminating process (S164), and then returns control to S101 in FIG. 8.

### <Example of Flow of Processing of Portable Terminals 41 to 44>

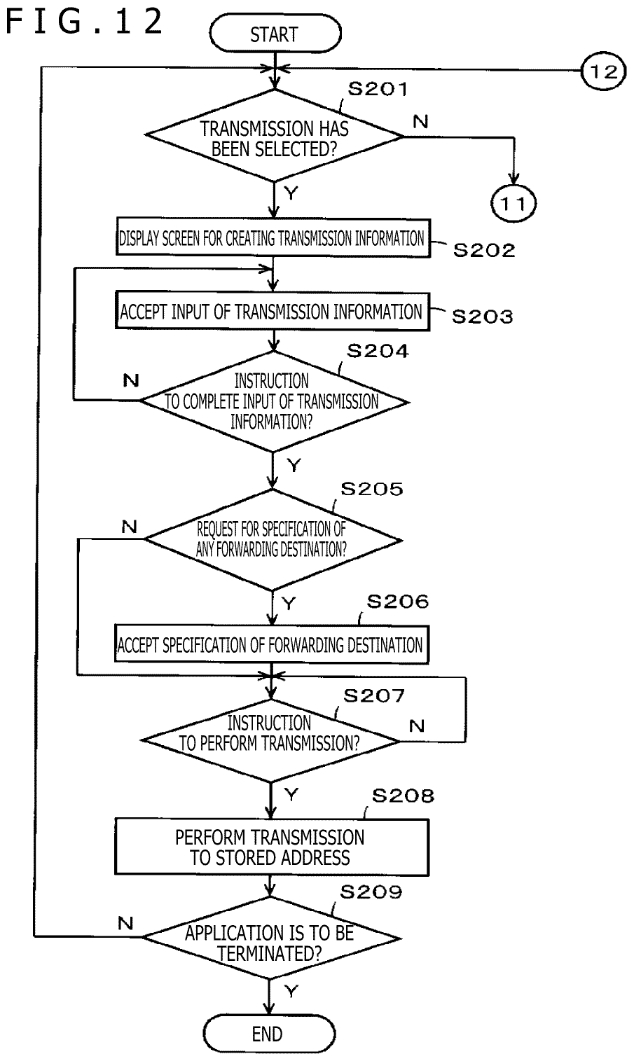

FIGS. 12 to 14 are diagrams for explaining an example of a flow of a processing operation performed by the communication control processing circuit 409 in each of the portable terminals 41 to 44 that perform communication with the information processing apparatus 1 according to this embodiment. Processing of the communication control processing circuit 409 is performed by an application program installed in advance on each of the portable terminals 41 to 44, and the processing is started at “START” in FIG. 12 when the application program is started by the user. It is assumed in the following description that the control circuit 401 in the configuration of FIG. 6 performs the processing.

The control circuit 401 determines whether or not transmission of an electronic mail to the information processing apparatus 1 has been selected by the user (S201 in FIG. 12). If it is determined at S201 that transmission of an electronic mail has been selected, the control circuit 401 displays a screen for creating transmission information of the electronic mail on the display screen of the display device 410 (S202), and accepts input by the user of the transmission information via the touch panel 411 (S203).

Then, the control circuit 401 determines whether or not an instruction to complete the input of the transmission information has been received from the user (S204), and if it is determined that such an instruction has not been received, the control circuit 401 returns control to S203, and continues to accept the input of the transmission information until the instruction to complete the input of the transmission information is received from the user.

If it is determined at S204 that the instruction to complete the input of the transmission information has been received from the user, the control circuit 401 inquires whether or not a forwarding destination will be specified, waits for a response thereto from the user, and determines whether or not there is a request for specification of any forwarding destination (S205). If it is determined at S205 that there is a request for specification of any forwarding destination, the control circuit 401, for example, displays a list of possible destinations that can be specified as a forwarding destination, and accepts specification of a forwarding destination (S206).

Then, the control circuit 401 waits to accept an instruction to perform transmission from the user (S207), and if it is determined that an instruction to perform transmission has been accepted, the control circuit 401 transmits an electronic mail to the stored address of the information processing apparatus 1 (S208).

Meanwhile, if it is determined at S205 that there is not a request for specification of any forwarding destination, the control circuit 401 proceeds to S207, skipping the process of S206, and waits to accept an instruction to perform transmission from the user, and if it is determined that an instruction to perform transmission has been accepted, the control circuit 401 transmits an electronic mail to the stored address of the information processing apparatus 1 at S208.

Subsequent to S208, the control circuit 401 inquires of the user whether or not the application program for the communication control processing circuit 409 is to be terminated, and makes a determination by waiting for a response thereto (S209), and if there is not a response indicating that the application program is to be terminated, the control circuit 401 returns control to S201, whereas if a response indicating that the application program is to be terminated is received, the control circuit 401 ends this processing routine.

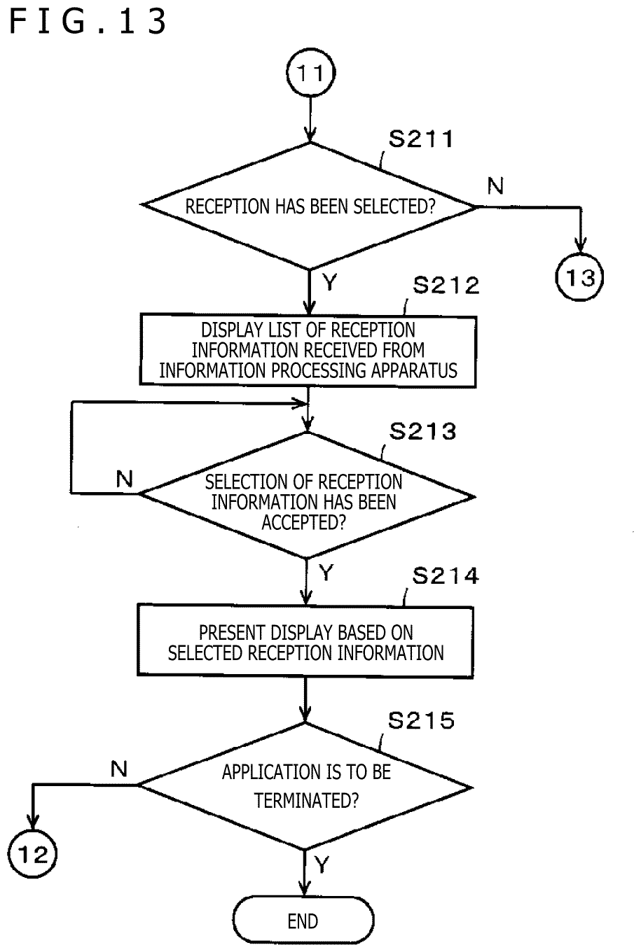

Meanwhile, if it is determined at S201 that transmission has not been selected, the control circuit 401 determines whether or not reception of an electronic mail has been selected (S211 in FIG. 13), and if it is determined that reception of an electronic mail has been selected, the control circuit 401 displays a list of reception information of electronic mails received from the information processing apparatus 1 (S212). Next, the control circuit 401 waits to accept selection by the user of reception information from the list, and if it is determined that selection of reception information has been accepted, the control circuit 401 presents a display based on the selected reception information on the display screen of the display device 410 (S214).

Then, the control circuit 401 inquires of the user whether or not the application program for the communication control processing circuit 409 is to be terminated, and makes a determination by waiting for a response thereto (S215), and if there is not a response indicating that the application program is to be terminated, the control circuit 401 returns control to S201, whereas if a response indicating that the application program is to be terminated is received, the control circuit 401 ends this processing routine.

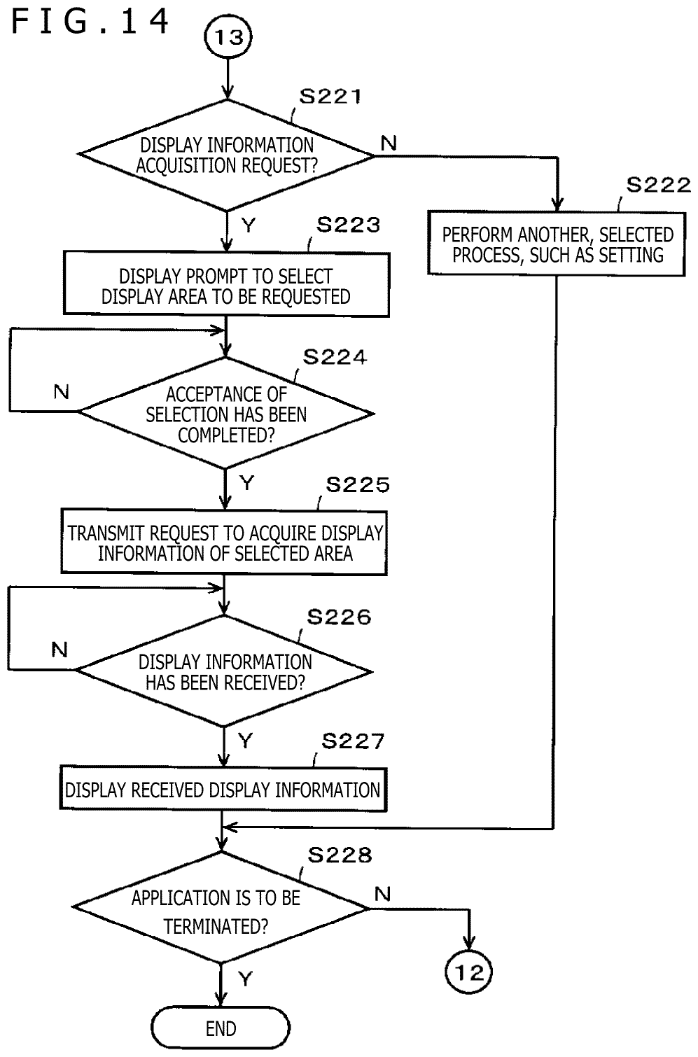

Meanwhile, if it is determined at S211 that reception of an electronic mail has not been selected, the control circuit 401 determines whether or not a display information acquisition request has been selected (S221 in FIG. 14). If it is determined at in S221 that a display information acquisition request has not been selected, the control circuit 401 performs another, selected process, such as setting (S222). Thereafter, the control circuit 401 inquires of the user whether or not the application program for the communication control processing circuit 409 is to be terminated, and makes a determination by waiting for a response thereto (S228), and if there is not a response indicating that the application program is to be terminated, the control circuit 401 returns control to S201, whereas if a response indicating that the application program is to be terminated is received, the control circuit 401 ends this processing routine.

If it is determined at S221 that a display information acquisition request has been selected, the control circuit 401 displays a list of areas of display information that can be requested and a prompt to select an area therefrom on the display screen of the display device 410 (S223), and waits for completion of acceptance of the selection (S224). Then, if it is determined at S224 that acceptance of the area of display information to be requested has been completed, the control circuit 401 transmits a display information acquisition request for the selected area to the information processing apparatus 1 (S225).

The control circuit 401 waits to receive display information that is to be sent in response to the transmitted display information acquisition request (S226), and, if reception of the display information is confirmed at S226, the control circuit 401 displays the received display information on the display screen of the display device 410 (S227). Thereafter, the control circuit 401 inquires of the user whether or not the application program for the communication control processing circuit 409 is to be terminated, and makes a determination by waiting for a response thereto (S228), and if there is not a response indicating that the application program is to be terminated, the control circuit 401 returns control to S201, whereas if a response indicating that the application program is to be terminated is received, the control circuit 401 ends this processing routine.

### <Example of Flow of Processing of Portable Terminals 45K and 46K>

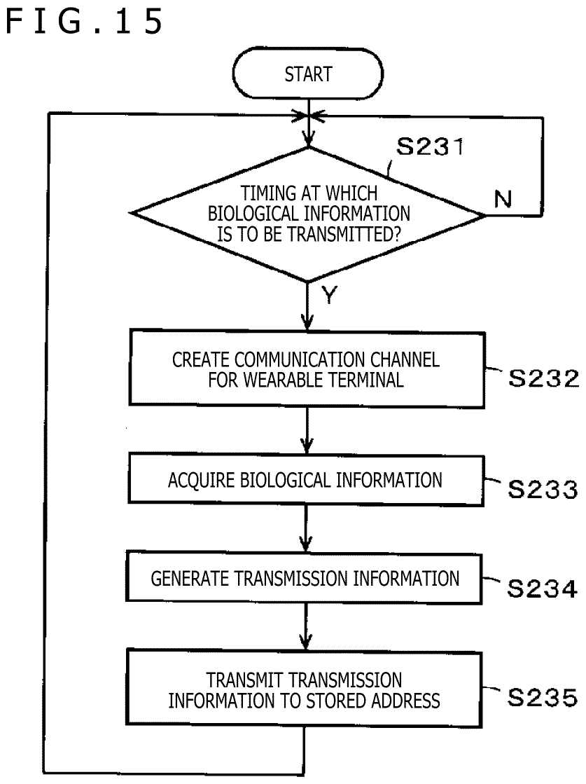

FIG. 15 is a diagram for explaining an example of a flow of a processing operation performed by the communication control processing circuit 409 in each of the portable terminals 45K and 46K that perform communication with the information processing apparatus 1 according to this embodiment. Processing of the communication control processing circuit 409 is performed by an application program installed in advance on each of the portable terminals 45K and 46K, and the processing is started at “START” in FIG. 15 when the application program is started by the user. It is assumed in the following description that the control circuit 401 in the configuration of FIG. 6 performs the processing.

The control circuit 401 constantly monitors whether a timing at which biological information is to be transmitted has come (S231). Then, if it is determined at S231 that a timing at which the biological information is to be transmitted has come, the control circuit 401 creates a communication channel for the wearable terminal 45W or 46W that is configured to communicate with the short-range wireless communication circuit 408 (S232), and acquires the biological information regarding the person to be watched over from the wearable terminal 45W or 46W (S233).

Next, the control circuit 401 generates transmission information including the biological information (S234), and transmits an electronic mail including the generated transmission information to the stored address of the information processing apparatus 1 (S235). Then, the control circuit 401 returns control to S231, and repeats the processing of S231 and the subsequent processing.

### Advantageous Effects of Embodiment

As described above, in the information processing apparatus 1 according to the above-described embodiment, the display area of the display screen 11D is configured to include the partial display areas AR1 to AR7 respectively associated with communication addresses of the portable terminals 41 to 44, 45K, and 46K which are set and registered in advance as apparatuses that communicate with the information processing apparatus 1. Further, in each of the partial display areas AR1 to AR7, the reception information from the corresponding one of the portable terminals 41 to 44, 45K, and 46K with which the partial display areas AR1 to AR7 are associated is displayed. In addition, the detection area of the position detection sensor 13 is configured to include the partial input areas corresponding to the respective partial display areas AR1 to AR7, and the writing data of the writing input onto each partial input area is transmitted to that one of the portable terminals 41 to 44, 45K, and 46K which is associated with the corresponding one of the partial display areas AR1 to AR7.

Accordingly, convenience is provided in that, only by viewing each of the partial display areas AR1 to AR7 on the display screen 11D of the information processing apparatus 1, the reception information from the user of the corresponding one of the portable terminals 41 to 44, 45K, and 46K associated with the respective partial display areas AR1 to AR7 can be checked. In addition, great convenience is provided in that information to be transmitted to each of the users of the portable terminals 41 to 44, 45K, and 46K associated with the partial display areas AR1 to AR7 can be transmitted to the user by making writing input of the information onto the corresponding one of the partial input areas corresponding to the partial display areas AR1 to AR7.

Further, the information processing apparatus 1 according to the above-described embodiment provides great convenience in that any of the partial display areas can be set as the common area, and the writing data of writing input onto the common area is transmitted to all of the portable terminals 41 to 44, 45K, and 46K set and registered in advance. In addition, an advantageous effect is provided in that any of the partial display areas can be used for the watching use, and this partial display area can be used to display the biological information regarding the person to be watched over, to achieve the watching purpose.

Moreover, the information processing apparatus 1 according to the above-described embodiment provides an advantageous effect in that, in response to a display information acquisition request from any portable terminal, not only the display information of the partial display area corresponding to that portable terminal but also the display information of another partial display area(s) or the display information of the whole of the display screen 11D can be transmitted to the portable terminal that has sent the display information acquisition request.

### OTHER EMBODIMENTS OR MODIFICATIONS

While it has been assumed in the foregoing description that information capable of being displayed, such as text information and image information, is communicated between the information processing apparatus 1 according to the above-described embodiment and the portable terminals, it is needless to say that audio information may also be communicated therebetween. In that case, the information processing apparatus 1 may be provided with a circuit for having a telephone conversation with a telephone function of a portable terminal, and may be configured to have functions of a telephone transmitter (i.e., a microphone) and a telephone receiver (i.e., a speaker).

In addition, the information processing apparatus 1 according to the above-described embodiment is provided with the two wireless communication circuits 171 and 172 to communicate with the portable terminals. As such, the two wireless communication circuits 171 and 172 can be used to implement communication of information between two portable terminals through the information processing apparatus 1. Suppose, for example, that the “father” is communicating with the information processing apparatus 1 through the wireless communication circuit 171 with use of the portable terminal 41, and desires to communicate with, for example, “Taro” and makes a request to the information processing apparatus 1 for “Taro” as a person to communicate with. Then, the information processing apparatus 1 can create a communication channel and connect to the portable terminal 44 of “Taro” through the unoccupied wireless communication circuit 172 to transmit information sent from the portable terminal 41 of the “father” to the portable terminal 44 of “Taro,” or to transmit information sent from the portable terminal 44 of “Taro” to the portable terminal 41 of the “father.”

In the information processing apparatus 1 according to the above-described embodiment, the marker-attached position indicator 2 is used as a position indicator for indicating a position on the position detection sensor 13, and the transparent panel is provided on the display screen 11D. However, the position indicator may not have a function of making ink writing on the front surface 12a of the transparent panel 12.

While the position detection sensor used in the above-described embodiment is of an electromagnetic inductive coupling type, a position detection sensor of a capacitive coupling type may alternatively be used in conjunction with, for example, a position indicator of an active capacitive type, which transmits a signal for position detection to the position detection sensor.

While it has been assumed in the foregoing description that electronic mail communication is performed between the information processing apparatus 1 according to the above-described embodiment and the portable terminals, it is needless to say that communication between an information processing apparatus according to an embodiment of this disclosure and a portable terminal is not limited to the electronic mail communication.

The portable terminals are not limited to high-performance mobile phone terminals, and any portable terminal including a display device and a control circuit and having a communication function may alternatively be used.

It is to be noted that the embodiment of the present disclosure is not limited to the foregoing embodiment, and that various changes can be made without departing from the spirit of the present disclosure.

What is claimed is:

1. An information processing apparatus comprising:
a display device including a display screen;
a communication circuit which, in operation, exchanges information with a plurality of portable terminals; and
a control circuit connected to the display device and the communication circuit,
wherein the display screen includes a display area having a plurality of partial display areas in a plurality of separate regions of the display screen, each of the partial display areas being associated with a different one of the portable terminals, and
wherein the control circuit, in operation, displays, in each partial display area of the partial display areas, information communicated via the communication circuit with the one of the portable terminals associated with the partial display area.

2. The information processing apparatus according to claim 1, further comprising:
a memory storing communication address information including a plurality of addresses used to communicate with the portable terminals via the communication circuit, wherein each of the addresses is associated with a corresponding one of the partial display areas.

3. The information processing apparatus according to claim 1, wherein the control circuit, in operation, displays a display image based on information acquired from each of the portable terminals via the communication circuit, and wherein the display image in each partial display area of the partial display areas displays is based on the information acquired from the one of the portable terminals associated with the partial display area.

4. The information processing apparatus according to claim 3, wherein, when the information acquired from one of the portable terminals via the communication circuit includes a forwarding request to forward the information to one or more of the portable terminals associated with one or more of the partial display areas, the control circuit forwards the information acquired via the communication circuit to the one or more of the portable terminals associated with the one or more of the partial display areas specified by the forwarding request.

5. The information processing apparatus according to claim 1, wherein, when an acquisition request to acquire display information has been received from one of the portable terminals via the communication circuit, the control circuit transmits the display information of the one of the partial display areas associated with the one of the portable terminals that has sent the acquisition request to the one of the portable terminal that has sent the acquisition request.

6. The information processing apparatus according to claim 1, wherein, when an acquisition request to acquire display information of a specified one of the partial display areas has been received from one of the portable terminals via the communication circuit, the control circuit transmits the display information of the specified partial display area to the one of the portable terminals that has sent the acquisition request.

7. The information processing apparatus according to claim 1, wherein, when an acquisition request to acquire display information of two or more of the partial display areas has been received from one of the portable terminals via the communication circuit, the control circuit transmits the display information of the two or more partial display areas specified by the acquisition request to the one of the portable terminals that has sent the acquisition request.

8. The information processing apparatus according to claim 1, wherein, when an acquisition request to acquire display information of a whole of the display screen of the display device has been received from one of the portable terminals via the communication circuit, the control circuit transmits the display information of the whole of the display screen to the one of the portable terminals that has sent the acquisition request.

9. The information processing apparatus according to claim 1, when information from one of the portable terminals has been received, the control circuit displays, on the one of the partial display areas associated with the one of the portable terminals, the information received from the one of the portable terminals, in place of information received earlier or in addition to the information received earlier.

10. The information processing apparatus according to claim 9, wherein:
the one of the portable terminals associated with the one of the partial display areas collects biological information regarding a person wearing a wearable terminal and transmits the biological information, and
the biological information regarding the person wearing the wearable terminal is displayed on the one of the partial display areas.

11. The information processing apparatus according to claim 1, further comprising:
a plurality of individual storage areas each corresponding to a separate one of the partial display areas,
wherein reception information received from each portable terminal of the portable terminals is stored in one of the individual storage areas corresponding to the one of the partial display areas associated with the portable terminal.

12. The information processing apparatus according to claim 1, further comprising:
a position detection circuit which, in operation, detects a position indicated by a position indicator,
wherein the position indicated by the position indicator and detected by the position detection circuit is associated with the display area of the display screen.

13. The information processing apparatus according to claim 12, wherein the position detection circuit includes a position detection sensor that overlaps with the display screen of the display device, and the display area of the display screen forms an input area for the position indicated by the position indicator on the position detection sensor.

14. The information processing apparatus according to claim 13, wherein:
the input area of the position detection sensor includes a plurality of partial input areas corresponding to and overlapping with the plurality of partial display areas of the display screen, and,
when input of a position indicated by the position indicator onto one of the plurality of partial input areas has been detected by the position detection circuit, the control circuit transmits information of the input made by the position indicator and detected by the position detection circuit to one of the portable terminals associated with one of the partial display areas corresponding to the one of the partial input areas.

15. The information processing apparatus according to claim 14, wherein,
the control circuit transmits the information of the input made by the position indicator and detected by the position detection circuit to the one of the portable terminals associated with the one of the partial display areas corresponding to the one of the partial input areas, and also to another one of portable terminals specified by a request for transmission information.

16. The information processing apparatus according to claim 14, wherein:
the input area of the position detection sensor includes, in addition to the plurality of partial input areas, a common input area overlapping with a common display area different from the plurality of partial display areas, and,
when input of a position indicated by the position indicator onto the common input area has been detected by the position detection circuit, the control circuit transmits information of the input made by the position indicator and detected by the position detection circuit to each of the portable terminals.

17. The information processing apparatus according to claim 13, wherein:
an upper surface of the display screen has a transparent cover member disposed thereon, and
the position indicator includes a writing portion which, in operation, dispenses ink on the cover member and an interaction portion which, in operation, exchanges a signal with the position detection sensor.

18. The information processing apparatus according to claim 14, further comprising:
a plurality of individual storage areas each corresponding to a separate one of the partial display areas,
wherein, when input of a position indicated by the position indicator onto one of the plurality of partial input areas has been detected by the position detection circuit, the control circuit stores information of the input made by the position indicator and detected by the position detection circuit in one of the individual storage area corresponding the one of the plurality of partial input areas.

19. The information processing apparatus according to claim 12, wherein the position detection circuit detects the position indicated by the position indicator, through electromagnetic inductive coupling with the position indicator.

20. The information processing apparatus according to claim 12, wherein the position detection circuit detects the position indicated by the position indicator, through capacitive coupling with the position indicator.
